(19) United States
(12) Patent Application Publication
(10) Pub. No.: US 2023/0080348 A1
(43) Pub. Date: Mar. 16, 2023

(54) MACHINE LEARNING TECHNIQUES FOR DETECTING REDUCED BLOOD FLOW CONDITIONS

(71) Applicant: University of Pittsburgh - Of the Commonwealth System of Higher Education, Pittsburgh, PA (US)

(72) Inventors: Shyam Visweswaran, Pittsburgh, PA (US); Jeremy U. Espino, Pittsburgh, PA (US); Kayhan Batmanghelich, Pittsburgh, PA (US); Parthasarathy D. Thirumala, Glenshaw, PA (US); Amir Mina, Pittsburgh, PA (US)

(21) Appl. No.: 17/930,854

(22) Filed: Sep. 9, 2022

Related U.S. Application Data
(60) Provisional application No. 63/242,980, filed on Sep. 10, 2021.

(51) Int. Cl. G16H 50/20; G16H 40/63

(52) U.S. Cl. CPC G16H 50/20; G16H 40/63

(57) ABSTRACT

This document describes machine learning techniques for detecting reduced blood flow conditions, such as ischemia and stroke, in real-time based on electroencephalography (EEG) signals. In one aspect, a method includes receiving patient data that includes a set of EEG signals generated by an EEG device measuring brain function of the patient. A set of feature values are generated for the patient using the EEG signals. The feature values are provided as input to a trained machine learning model that has been trained to detect reduced blood flow conditions of patients. An indication of whether the patient has the reduced blood flow condition is received as a machine learning output of the trained machine learning model. The indication of whether the patient has the reduced blood flow condition is provided.

### CROSS-REFERENCE TO RELATED APPLICATION

This application claims priority to, and the benefit of, U.S. Provisional Application No. 63/242,980, filed Sep. 10, 2021, titled “MACHINE LEARNING TECHNIQUES FOR DETECTING REDUCED BLOOD FLOW CONDITIONS”, which is incorporated herein by reference.

### TECHNICAL FIELD

This specification relates to data processing and machine learning techniques for detecting reduced blood flow conditions, such as ischemia and stroke, using electroencephalography (EEG) signals.

### BACKGROUND

Reduced blood flow conditions, such as ischemia and stroke, that occur during surgery are devastating complications that can result in death, disability, and other burdens to patients and their families.

### SUMMARY

This specification generally describes machine learning techniques for detecting reduced blood flow conditions based on EEG signals. During surgery, other medical procedures, and for other reasons, patients can be continuously monitored using an EEG device, which electrically measures brain function. The EEG signals can be provided as input to a machine learning model that is trained to detect one or more reduced blood flow conditions, such as ischemia, stroke, or both. The machine learning model can detect changes and other features in EEG signals in real-time or near real-time (e.g., within one second or less) that are indicative of ischemia and/or stroke and output a score that represents a likelihood that the patient is experiencing a reduced blood flow condition. If a reduced blood flow condition is detected, a blood flow condition monitoring device can alert medical staff. For example, the blood flow condition monitoring device can receive the EEG signals in real-time during surgery, during another type of medical procedure, or while otherwise monitoring a patient in any setting, analyze the EEG signals as they are received, and alert medical staff upon detection of a reduced blood flow condition. The machine learning model can output a score that represents a likelihood that the patient is experiencing a reduced blood flow condition based on features extracted from the EEG signals. The blood flow monitoring device can compare the score to one or more thresholds and alert medical staff if the score satisfies (e.g., meets or exceeds) a threshold indicative of the reduced blood flow condition.

The reduced blood flow condition monitoring device can include a data formatting engine that rapidly processes EEG signals and converts the EEG signals into formats that are machine processable to enable real-time detection of reduced blood flow conditions. The data formatting engine can employ memory management techniques that enable the data formatting engine to quickly identify new EEG signal data in a data storage location to reduce the latency in converting newly generated EEG signals to inputs for the machine learning model, which reduces the latency in detecting reduced blood flow conditions.

In general, one innovative aspect of the subject matter described in this specification can be embodied in methods that include the actions of receiving patient data including a set of EEG signals generated by an EEG device measuring brain function of the patient; generating, using the EEG signals, a set of feature values for features of the patient; providing the feature values as input to a trained machine learning model that has been trained to detect reduced blood flow conditions of patients; receiving, as a machine learning output of the trained machine learning model, an indication of whether the patient has the reduced blood flow condition; and providing the indication of whether the patient has the reduced blood flow condition. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The foregoing and other embodiments can each optionally include one or more of the following features, alone or in combination. In some aspects, the reduced blood flow condition includes one of ischemia or a stroke. Some aspects include determining that the patient has the reduced blood flow condition based on the machine learning output. Providing the indication of whether the patient has the reduced blood flow condition can include sending an alert to one or more medical professionals.

In some aspects, patient data includes one or more notes indicating medical observations related to the patient that were made by a clinician. The one or more notes can include a note that indicates a time when a carotid artery is clamped during a medical procedure being performed on the patient.

In some aspects, the trained machine learning model is trained based on historical patient data for multiple patients. The historical data for each individual patient can include sequences of EEG signals for the individual patient that were monitored during a medical procedure being performed on the individual patient, one or more medical professional notes generated by a medical professional during the medical procedure, and annotations indicating when, relative to the sequences of EEG signals, a reduced blood flow condition was detected for the individual patient during the medical procedure.

In some aspects, the feature values include a set of ratios between power values of a first frequency band of the EEG signals and corresponding power values of a second frequency band of the EEG signals. The feature values can include a ratio between (i) a first sum of a first power value of a first frequency band of the EEG signals and a second power value of a second frequency band of the EEG signals and (ii) a second sum of a third power value of a third frequency band of the EEG signals and a fourth power value of a fourth frequency band of the EEG signals.

In some aspects, the feature values include a difference between a highest voltage among the EEG signals and a lowest voltage among the EEG signals. The feature values can include a particular frequency at which a specified percentage of power values of the EEG signals is at or lower than the particular frequency. Generating the feature values can include generating a set of feature values in real-time for each second of EEG signals received from an EEG device connected to the patient.

In general, another innovative aspect of the subject matter described in this specification can be embodied in methods that include the actions of receiving patient data comprising a set of EEG signals generated by an EEG device measuring brain function of the patient; generating, using the EEG signals, a set of feature values for features of the patient; providing the feature values as input to a trained machine learning model that has been trained to detect reduced blood flow conditions of patients; receiving, as a machine learning output of the trained machine learning model, an indication of whether the patient has the reduced blood flow condition; and providing the indication of whether the patient has the reduced blood flow condition. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The foregoing and other embodiments can each optionally include one or more of the following features, alone or in combination. In some aspects, the reduced blood flow condition includes one of ischemia or a stroke.

Some aspects include determining that the patient has the reduced blood flow condition based on the machine learning output. Providing the indication of whether the patient has the reduced blood flow condition can include sending an alert to one or more medical professionals.

In some aspects, the trained machine learning model is trained based on historical patient data for multiple patients. The historical data for each individual patient can include sequences of EEG signals for the individual patient that were monitored during a medical procedure being performed on the individual patient, one or more medical professional notes generated by a medical professional during the medical procedure, and annotations indicating when, relative to the sequences of EEG signals, a reduced blood flow condition was detected for the individual patient during the medical procedure.

In some aspects, the feature values includes a set of ratios between power values of a first frequency band of the EEG signals and corresponding power values of a second frequency band of the EEG signals. In some aspects, the feature values include a ratio between (i) a first sum of a first power value of a first frequency band of the EEG signals and a second power value of a second frequency band of the EEG signals and (ii) a second sum of a third power value of a third frequency band of the EEG signals and a fourth power value of a fourth frequency band of the EEG signals.

In some aspects, the feature values include a difference between a highest voltage among the EEG signals and a lowest voltage among the EEG signals. The feature values can include a particular frequency at which a specified percentage of power values of the EEG signals is at or lower than the particular frequency.

In some aspects, generating the feature values includes generating a set of feature values in real-time for each second of EEG signals received from an EEG device connected to the patient. Each set of feature values can include feature values for a time period beginning a specified amount of time before the second for which the set of feature values is generated. The specified amount of time can be 20 seconds.

In some aspects, the patient data includes a set of data files that each include different formats of data including multiple data files comprising data for the set of EEG signals. Some aspects include reprocessing the data of each data file to convert the data of each data file to a same standard format. Some aspects include maintaining each data file in an open state throughout a time period in which the patient is being monitored and, for each data file, continuously or periodically scanning memory locations at which data for each data file is stored to acquire any new data written to the memory locations. Continuously or periodically scanning memory locations at which data for each data file is stored to acquire any new data written to the memory locations can include monitoring a flag that indicates an end of file location in memory for the data file.

The subject matter described in this specification can be implemented in particular embodiments and may result in one or more of the following advantages. The machine learning models described in this document can more accurately and more quickly detect reduced blood flow conditions that occur in patients as compared to medical professionals, e.g., neurophysiologists. The machine learning models can be trained on a variety of features for which changes in the features can be indicative of reduced blood flow conditions. As such features (especially those that are based on multiple EEG signals) may not be readily detectable by medical staff, the machine learning models can detect reduced blood flow conditions that medical staff members are unable to detect in real-time, thereby greatly reducing the amount of time required to diagnose such conditions and therefore greatly reducing the impact of prolonged ischemia and/or stroke that occur during surgery or otherwise. Currently, medical staff members monitor the raw EEG signals and selected EEG power information and additional information derived from the EEG power information. However, a human can evaluate only a handful of features over 10-20 second time periods while the machine learning models described in this document can evaluate hundreds of features over very short durations, e.g., one half to one second time periods. Further, subtle changes in EEG signals, such as a 10% reduction in value, and complex EEG features such as the ratio of two simpler features, that are indicative of ischemia, are not readily detected by medical staff. Thus, the machine learning models described in this document can reliably evaluate more features, over shorter durations, detect subtler changes, and consider complex features compared to medical staff. The machine learning models described in this document can also be used to identify a small set of useful features that are indicative of a reduced blood flow state from a very large number of possible EEG features that can be created. Using a smaller set of key features can reduce the number of features that need to be calculated, which in turn enables these features to be calculated for even shorter durations than one second thus leading to increased speed and efficiency of detecting a reduced blood flow state. A data formatting engine can further reduce the amount of time to diagnose a reduced blood flow condition by rapidly converting EEG signals and note files into a machine processable format and by using memory management techniques to quickly identify new EEG signal data for the patient.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A is an example of an environment in which a blood flow condition monitoring device detects reduced blood flow conditions of patients.

FIG. 1B is another example of an environment in which a blood flow condition monitoring device detects reduced blood flow conditions of patients.

FIG. 2 is a block diagram of an example data formatting engine that converts data of multiple different formats into a standard format.

FIG. 3 is a flow diagram of an example process for training a machine learning model to detect reduced blood flow conditions of patients.

FIG. 4 is a flow diagram of an example process for detecting reduced blood flow conditions in patients using a trained machine learning model.

FIG. 5 is a block diagram of a computing system that can be used in connection with computer-implemented methods described in this document.

Like reference numbers and designations in the various drawings indicate like elements.

### DETAILED DESCRIPTION

This specification generally describes machine learning techniques, including devices that implement such techniques, for detecting reduced blood flow conditions based on electroencephalography (EEG) signals. An estimated 50,000 patients per year experience a stroke during surgery, leading to 10,000 deaths and disability for those who survive. Life-saving clot removal is available but generally is only effective if the stroke can be detected early. The precursor of stroke is ischemia which results from a temporary decrease in blood flow to the brain, which can happen due to a drop in blood pressure or due to a blood clot that travels to the brain and blocks blood flow. Intraoperative neurophysiological monitoring (IONM) of brain function with EEG can be used by medical staff to identify both brain ischemia and stroke in real-time. However, there is a lack of automated methods for consistent, real-time intraoperative monitoring of EEG to detect brain ischemia and/or stroke. Currently, when an EEG is utilized during surgery, a trained neurophysiologist visually monitors the EEG to identify changes, and sometimes a single neurophysiologist needs to monitor several patients simultaneously. However, human visual monitoring can be unreliable and humans are unable to detect many features that are found in EEG signals and that are indicative of reduced blood flow conditions. In addition, trained personnel for visual monitoring are not available in all hospitals or other settings where reduced blood flow condition detection is useful, e.g., in standard patient rooms, ambulances, primary care offices, and non-medical facilities such as offices, sports/entertainment venues, etc. The systems and techniques described in this document evaluate EEG data for reduced blood flow conditions such as intraoperative brain ischemia and stroke using a trained machine learning model. This enables real-time, accurate, and consistent detection of reduced blood flow conditions, including ischemia and stroke.

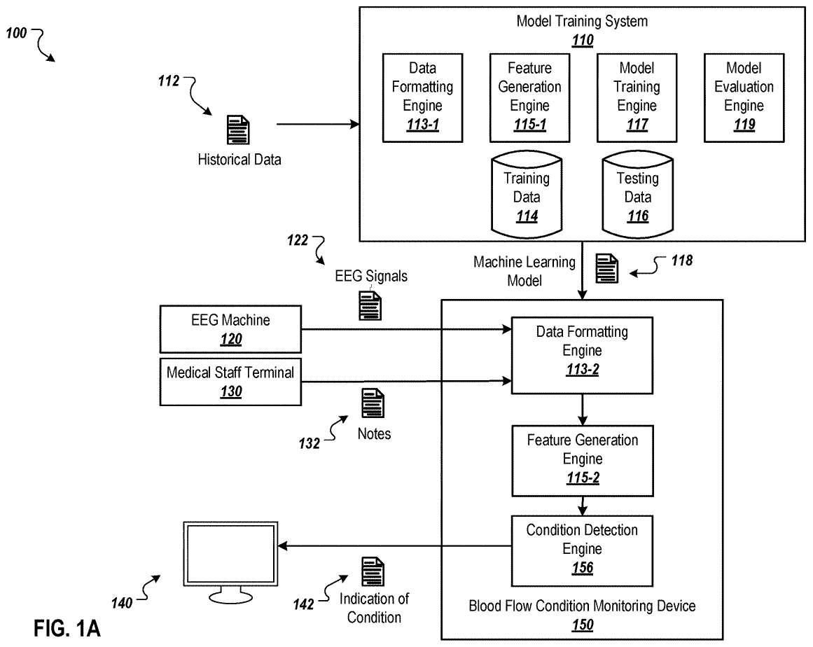

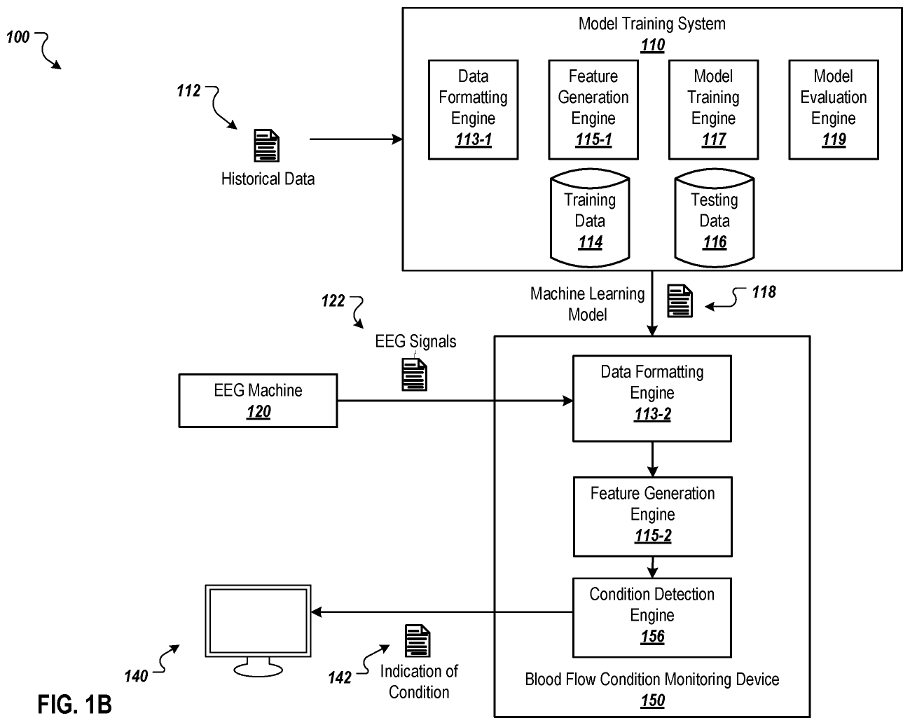

FIG. 1A is an example of an environment 100 in which a blood flow condition monitoring device 150 detects reduced blood flow conditions of patients. The reduced blood flow conditions can include conditions in which an inadequate blood supply is provided to an organ of the patient's bodies, such as ischemia and/or stroke.

The example environment 100 also includes a model training system 110 that is configured to train machine learning models to detect the reduced blood flow conditions based on EEG signals. Although shown as part of the environment 100, the model training system 110 may or may not be part of a medical or non-medical environment in which the blood flow condition monitoring device 150 is deployed. Instead, the machine learning model(s) can be preinstalled on the blood flow condition monitoring device 150 and/or received over a network or by way of a memory storage device, e.g., a universal serial bus (USB) memory stick. For example, the model training system 110 can be in a different location than the blood flow condition monitoring device 150 and the blood flow condition monitoring device 150 can receive the machine learning model(s) via a network connection to the model training system 110. The model training system 110 can include one or more computers in one or more locations.

The model training system 110 can train the machine learning models using historical data 112 for a set of patients. The historical data 112 can include, for each patient, EEG signals measured by an EEG machine connected to the patient, e.g., during a medical procedure such as a surgery being performed on the patient. During the procedure, EEG signals are obtained by placing electrodes on the head of the patient.

For example, the electrodes can be placed on the head of the patient in eight standard locations that cover all brain regions. In this example, four electrodes are placed on the left side of the patient's head to cover the left hemisphere of the patient's brain and four electrodes are placed on the right side of the patient's head to cover the right hemisphere of the patient's brain.

On each side of the brain, one electrode covers the front of the brain (called the frontal region), two electrodes cover the side of the brain (referred to as the parietal and temporal regions), and one electrode covers the back of the brain (referred to as the occipital region). The eight electrodes have standard names: F3 (frontal region of the left hemisphere), P3 (parietal region of the left hemisphere), T3 (temporal region of the left hemisphere), O1 (occipital region of the left hemisphere), F4 (frontal region of right hemisphere), P4 (parietal region of right hemisphere), T4 (temporal region of right hemisphere), and O2 (occipital region of right hemisphere).

Each EEG signal, sometimes referred to as a “lead,” is measured as a difference in electrical potential or voltage between two electrodes; for example, the channel F3-T3 measures the difference in electrical voltage between the F3 and T3 electrodes. Clinically, EEG signals are decomposed into functionally distinct frequency bands that include a delta frequency band (1-3 Hz), a theta frequency band (4-7 Hz), an alpha frequency band (8-13 Hz), a beta frequency band (14-20 Hz), and a gamma frequency band (30-100 Hz).

The EEG data received by the model training system 110 can be in the form of streams of numbers, e.g., in binary format. For example, the EEG data can include a time series sequence of numbers for each frequency band of each pair of channels for which a difference in electrical voltage is measured, ordered in chronological time. As described in more detail below, these sequences of numbers can be used to generate feature values for features for training the machine learning models. In other implementations, the time-series sampled EEG signals themselves can be used to train the machine learning model. In this example, time-series sampled EEG signals can be provided as inputs to the machine learning model, e.g., in addition to or in place of the features derived from the EEG signals. Each EEG signal can have a corresponding timestamp that indicates a time at which the EEG signal was measured.

The historical data 112 can also include, for each patient, one or more notes indicating medical observations related to the patient. For example, a medical staff member, e.g., a technologist, nurse, or physician, can record notes indicating (or describing) events that occur during a medical procedure being performed on the patient. The historical data 112 for the notes can include a timestamp indicating a time at which the observation occurred, e.g., a time at which the event occurred. The timestamp enables the model training system 110 to correlate the event with the sequence of EEG signals, e.g., using the timestamps of the EEG signals. In a particular example in which the medical procedure is a carotid endarterectomy (CEA) surgery, the clinician's note can indicate the time when a clamp was applied to the diseased carotid artery. Briefly, during CEA, the diseased carotid artery is clamped, and after clamping, there is an increased risk of ischemia/stroke. The EEG signals for the 10 minutes after the clamp has been applied provides rich data for training the machine learning models since this is the period of increased risk for a reduced blood flow condition.

The historical data 112 can also include, for each patient, annotations generated by an expert neurophysiologist. The annotations can indicate which periods of the EEG are indicative of a reduced blood flow condition, e.g., ischemia or a stroke. For example, an annotation can indicate a time (or location within the sequence of EEG signals) at which the reduced blood flow condition started. Thus, the historical data 112 for a patient can include timestamped EEG signal data, medical staff notes with times at which events occurred, and annotations for the patient's EEG that indicate places in the EEG when a reduced blood flow condition occurred if one was detected as having occurred for that patient.

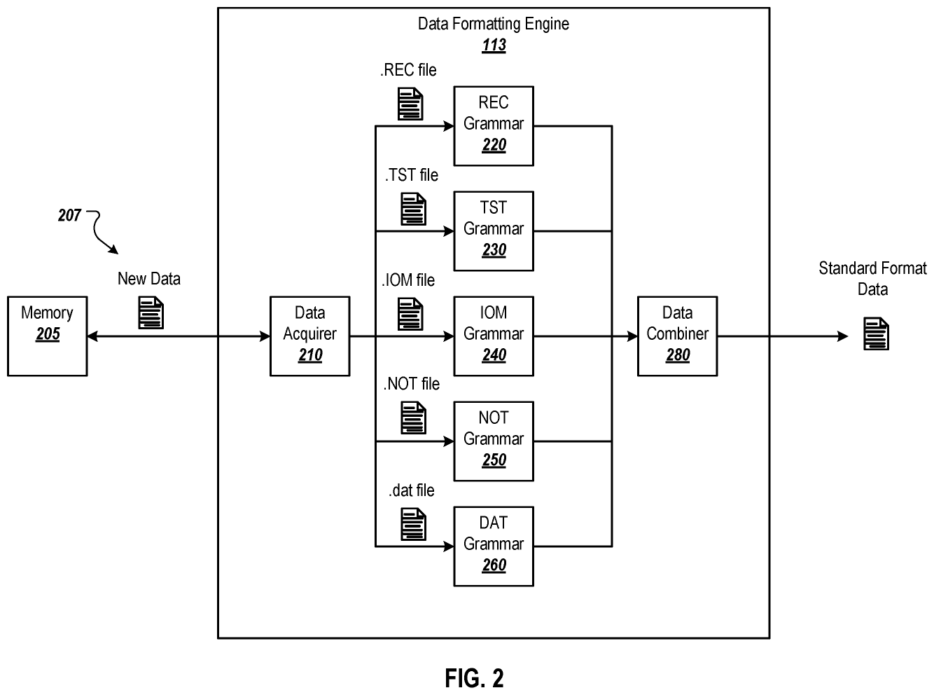

The model training system 110 includes a data formatting engine 113-1, a feature generation engine 115-1, a model training engine 117, and a model evaluation engine 119. The data formatting engine 113 can convert the historical data into a standard machine processable format. The standard format can be a format that can be processed by various applications. For example, the standard format can be JavaScript Object Notation (JSON) or a comma-separated values (CSV) file. An example data formatting engine 113 is shown in FIG. 2 and described below.

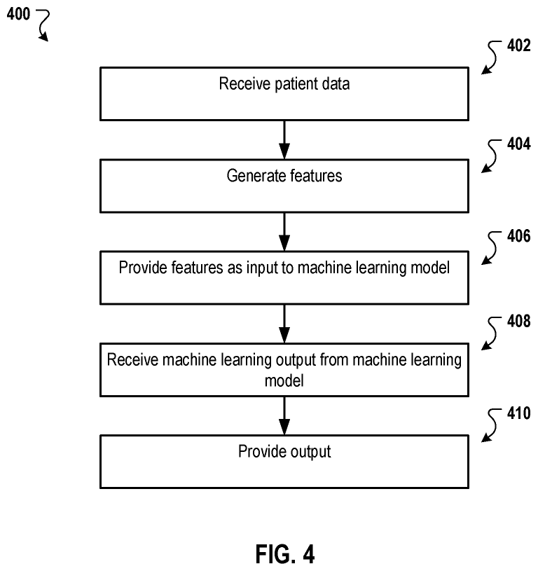

The feature generation engine 115-1 can generate feature values based on the historical data that has been formatted into the standard format and provide the feature values to the model training engine 119. An example set of features is described below with reference to FIG. 4.

The model training engine 117 can train one or more machine learning models using at least a portion of the historical data 112, e.g., using feature values that represent features of this portion of the historical data. In some implementations, the model training system 110 uses supervised learning techniques to train the machine learning models. For example, the model training engine 117 can generate feature values for a set of features using the EEG signals and the notes for the patients. In another example, the model training engine 117 can generate feature values for a set of features of using the EEG signals and the annotations generated by an expert neurophysiologist. In either example, these feature values can be referred to as the input variables for supervised learning. In this example, the annotation that indicates when the reduced blood flow condition is detected is the output variable, or label for the model training. Thus, supervised learning can be used to map the feature values that represent the EEG signals and the notes and/or annotations such that the machine learning models can output a score that represents a likelihood that a reduced blood flow condition is occurring based on input EEG signals and input notes and/or annotations. Example supervised learning models that can be trained by the model training system 110 include artificial neural networks (e.g., feedforward, convolutional, recurrent neural networks), logistic regression, naive Bayes classifiers, support vector classifiers, and random forest classifiers.

In some implementations, the model training engine 117 can use unsupervised learning techniques to train the machine learning models. Using unsupervised learning does not involve the use of labels, such that the annotations would not be required. Instead, the machine learning model discovers EEG signals or patterns that represent normal conditions (e.g., no reduced blood flow condition is present) and EEG signals or patterns that represent that a reduced blood flow condition is present. For instance, the model training engine 117 can identify one or more first clusters of feature values in one or more dimensions can and associate these clusters of feature values with one or more normal conditions. The model training engine 117 can also identify one or more second clusters of feature values in one or more dimensions and associate these clusters of feature values with one or more reduced blood flow conditions.

The data formatting engine 113 of the model training system 110 can split the historical data 112 into training data 114 and testing data 116. For example, the model training system 110 can include the historical data 112 for a first subset of the patients in the training data 114 and the historical data 112 for a second subset of the patients in the testing data 116. In a particular example, the first subset can include the historical data 112 for 50% of the patients and the second subset can include the historical data 112 for the other 50% of the patients. In another example, the first subset can include the historical data 112 for 75% of the patients and the second subset can include the historical data 112 for the other 25% of the patients. Other percentages can also be used.

The model training system 110 can train the machine learning models using the training data 114. After training the machine learning models, the model training system 110 can evaluate the models using the testing data 116. Testing a machine learning model can include providing the EEG signals for each patient of the testing data 116 as input to the machine learning model and comparing the prediction by the machine learning model to the annotation for the patient included on the testing data 116. Testing the machine learning models can also include generating a receiver operating characteristic (ROC) curve and determining the area under the ROC curve for each machine learning model. The machine learning model having the largest area under the ROC curve can be considered the best performing machine learning model.

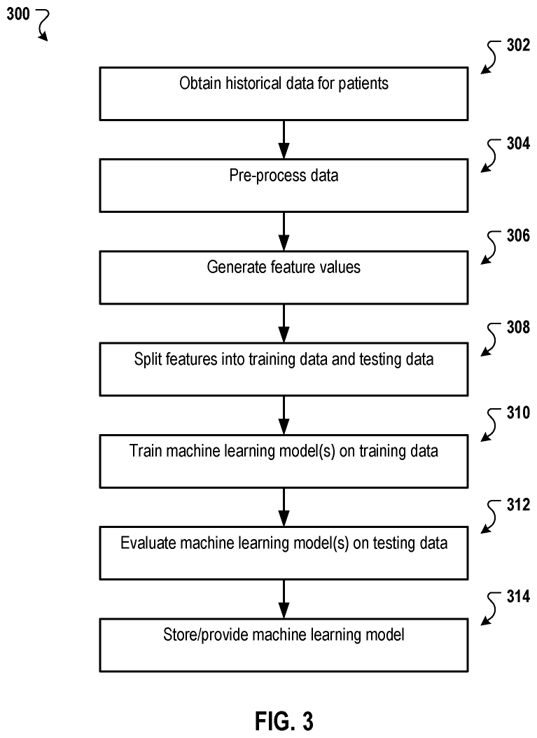

In some implementations, the model training system 110 can train multiple types of machine learning models using the training data 114 and evaluate each model using the test data 116. The model training system 110 can then select the best performing model (e.g., the one having the largest area under the ROC curve) as the machine learning model to use in the blood flow condition monitoring device 150. The selected machine learning model 118 can then be provided to the blood flow condition monitoring device 150, e.g., over a network or using a data storage device. An example process for training a machine learning model to detect reduced blood flow conditions is illustrated in FIG. 3 and described below.

The blood flow condition monitoring device 150 includes a data formatting engine 113-2, a feature generation engine 115-2, and a condition detection engine 156. The blood flow condition monitoring device 150 can be deployed universally and can be used to detect reduced blood flow conditions in any setting including in, for example, operating rooms, intensive care units (ICUs), standard patient rooms, ambulances, and non-medical settings. Although shown as a stand-alone device, the blood flow condition monitoring device 150 can be a component of another device, e.g., a component of an EEG device or other patient monitoring device. In another example, components of the blood flow condition monitoring device 150, e.g., the data formatting engine 113-2, the feature generating engine 115-2, and/or the condition detection engine 156, can be installed on an EEG device and can output probabilities and/or alerts using the display, speaker, or other component of the EEG device.

The blood flow condition monitoring device 150 receives EEG signals 122 of a patient from an EEG machine 120. For example, the blood flow condition monitoring device 150 can receive the EEG signals 122 in real-time while a medical procedure, e.g., surgery, is being performed on the patient or during other types of patient monitoring or diagnosis.

In this example, the blood flow condition monitoring device 150 can also receive notes 132 generated by a medical staff member using a medical staff terminal 130. The blood flow condition monitoring device 150 can also receive the notes in real-time as the notes are input to the terminal. In another example, as shown in FIG. 1B, the blood flow monitoring device 150 can receive EEG signals 122 without receiving notes 132.

The data formatting engine 113-2 can format the EEG signals and/or the notes in a manner suitable for processing by the feature generation engine 154, e.g., into the standard format. The data formatting engine 113-2 can be the same as the data formatting engine 113-1, which is described in more detail with reference to FIG. 2. The data formatting engine 152 can provide the formatted EEG signals and/or the notes to the feature generation engine 115-2.

The feature generation engine 115-2 can generate feature values for a set of features for input to the machine learning model 118 and provide the feature values to the condition detection engine 156 that includes the machine learning model 118. An example set of features is described below with reference to FIG. 4. The condition detection engine 156 can provide the feature values as input to the machine learning model 118 and receive machine learning outputs from the machine learning model 118. Each machine learning output can indicate a likelihood, e.g., a probability, that a reduced blood flow condition occurred within the time interval spanned by the EEG feature values that were provided as input to the machine learning model 118, as predicted according to the current values of the trainable parameters of the machine learning model 118. The machine learning output can be based on changes in the EEG signals within the time interval, occurrence of certain events reflected in clinician notes, or both. In some implementations, the machine learning output is provided as a score (e.g., a probability score) within a pre-defined scale of values, e.g., 0-1. In some implementations, the condition detection engine 156 compares the score to a threshold to make a classification decision such as “reduced blood flow condition detected” or “reduced blood flow condition not detected.” In some implementations, multiple thresholds are used. For example, the condition detection engine 156 can compare the score to a first threshold and a second threshold that is lower than the first threshold. If the score meets or exceeds the first threshold, the condition detection engine 156 can determine that a reduced blood flow condition is present. If the score is less than the second threshold, the condition detection engine 156 can determine that a reduced blood flow condition is not present. If the score is between the two thresholds, there may or may not be a reduced blood flow condition. An example process for using a trained machine learning model to detect reduced blood flow conditions is illustrated in FIG. 4 and described below.

The blood flow condition monitoring device 150 can provide an indication of whether a reduced blood flow condition has been detected. For example, the blood flow condition monitoring device 150 can provide an indication of whether the reduced blood flow condition has been detected to a monitor 140 located in the operating room or other area in which a medical procedure is being performed on the patient or the patient is otherwise being monitored. In this way, a medical staff member can view the monitor 140 in real-time during the procedure such that, if the reduced blood flow condition is detected using the machine learning model 118, the medical team can intervene to correct the condition. The blood flow condition monitoring device 150 can also indicate when the score is between the two thresholds described above so that the medical staff can determine whether to evaluate the patient further.

In another example, the blood flow condition monitoring device 150 can issue notifications, e.g., alerts, to medical professionals in response to detecting a reduced blood flow condition. For example, the blood flow condition monitoring device 150 can send, e.g., push, a notification to devices of a response team that is trained to intervene when such conditions are detected, e.g., when the score output by the machine learning model meets or exceeds a threshold indicative of a reduced blood flow condition.

The model training system 110 and blood flow condition monitoring device 150 can each be implemented locally, on-site at any location where EEG signals are obtained and/or surgeries are performed. In some implementations, however, all or portions of the training system 110 and/or blood flow condition monitoring device 150 may be implemented remotely from the location(s) where EEG signals are obtained or surgeries are performed. For example, blood flow monitoring device 150 may be deployed on remote servers and configured to process EEG signals and/or notes uploaded from a clinician's office. A real-time link can be maintained over one or more networks between the on-site computers (e.g., EEG machine 120 and medical staff terminal 130) and the remote flood flow condition monitoring device 150, such that EEG signals (and/or feature values) and/or notes are streamed to the monitoring device 150 for real-time processing, and condition indicators 142 generated by the condition detection engine 156 can be returned in real-time to the on-site machines. For example, likelihood scores or binary classifications indicating whether a reduced blood flow condition was detected can be generated and returned in real-time, and alerts/notifications can be generated according to one or more defined alerting/notification criteria.

FIG. 2 is a block diagram of an example data formatting engine 113 that converts data of multiple different formats into a standard format. The data formatting engine 113 includes a data acquirer 220, a set of grammar modules 220-260, and a data combiner 280.

The data acquirer 210 monitors for new data in memory 205 and retrieves any new data found in memory 205. One or more devices (e.g., an EEG machine 120 and/or medical staff terminal 130) can store EEG signals and associated files in the memory 205 as the data is measured or received. The device(s) can store the data in memory 205 in their native file formats. For example, an EEG machine can store EEG waveforms in a .REC file, store channel data in a .TST file, store patient data in a .IOM file, store medical staff notes in a .NOT file, and store timestamps of recordings in a .dat file.

The device(s) can be configured to store data in the memory in periodic increments, e.g., each second, every 500 ms, or based on another appropriate time period. Rather than read the entire file(s) stored in memory each time, the data acquirer 210 can use memory management techniques to reduce the amount of memory scanned when acquiring new data to process. Instead, each file can be kept open throughout the time that a patient is being monitored. For each file, the data acquirer 210 can monitor the memory location at which a last previous write to memory for that file was made. For example, the operating system of the EEG machine 120 may place an end of file (EOF) flag at the last memory location at which data for the file was written. The data acquirer 210 can continuously or periodically scan for the EOF flag at that memory location and, if the flag is removed, the data acquirer 210 can acquire data from subsequent memory locations until reaching the EOF flag again. This section of memory represents new data for that file. By monitoring the memory for each file in this way, the data acquirer 210 can retrieve new data as soon as it is written to memory by the EEG machine and thereby reduce the latency in processing the data so that the blood flow condition monitoring device 150 can rapidly detect reduced blood flow conditions based on the data.

For each type of file, the data acquirer 210 can provide the data to an appropriate grammar module 220-260. Each grammar module 220-260 can include a set of rules, e.g., a set of syntactic rules, for interpreting the data of a particular file type and converting the data to the standard format. In this example, the data formatting engine 113 includes an REC grammar module 220 for interpreting and converting .REC data, a TST grammar module 230 for interpreting and converting .TST data, an TOM grammar module 240 for interpreting and converting .IOM data, a NOT grammar module 250 for interpreting and converting .NOT data, and a DOT grammar module 260 for interpreting and converting .DAT data. Each grammar module 220-260 can be configured to convert its data as soon as new data is received from the data acquirer 210 and provide the converted data to the data combiner 280.

The data combiner 280 is configured to combine the data into a standard file in the standard data type, e.g., into a JSON file using JSON data. The data combiner 280 can use a template to place each type of data in a corresponding location in the file so that the data is readily processed by a feature generator and/or other device or engine.

FIG. 3 is a flow diagram of an example process 300 for training a machine learning model to detect reduced blood flow conditions of patients. In some implementations, the example process 300 can be performed by a machine learning model training system, such as the model training system 110 of FIG. 1. Operations of the process 300 can also be implemented as instructions stored on computer readable media, which may be non-transitory, and execution of the instructions by one or more data processing apparatus can cause the one or more data processing apparatus to perform the operations of the process 300. For brevity, the process 300 will be described in terms of a model training system.

The example process 300 is described in terms of using recorded, e.g., historical, data for patients during (CEA) surgeries as the 10 minutes after the clamp is applied to a diseased carotid artery provides rich data for training the machine learning model due to the increased risk of ischemia/stroke during that time period. However, the machine learning model can be trained using other data for patients that experienced reduced blood flow conditions and for which EEG data and other appropriate data has been recorded.

The data from many procedures can be used to train the machine learning model. In some implementations, the EEG data may not be filtered to remove noisy data or data from situations in which a lead of the EEG device was disconnected from the patient. In this way, the machine learning models are robust and account for real world conditions in which noise is present and leads become detached.

The model training system obtains data for a set of patients (302). The data can include historical data for patients that was recorded before, during, and/or after medical procedures performed on the patients. As described above, the historical data for a patient can include EEG signals recorded during a medical procedure being performed on the patient, one or more notes generated by a medical staff member (e.g., during the procedure), and annotations to the EEG to indicate when, if any, a reduced blood flow condition began or occurred during the medical procedure. Each piece of data can include one or more corresponding timestamps that each indicate a relevant time for the data. For example, the timestamp for an EEG signal can indicate a time at which the signal was measured. Each note for an event in a note file can include a timestamp indicating a time at which the event occurred or was observed. The timestamps for an annotation can indicate a time period (e.g., a start time and an end time) for an observed reduced blood flow event.

The EEG signals can be in the form of an EEG file that includes time series values, e.g., voltage levels, for each frequency band of each pair of channels for which a difference in electrical voltage is measured, ordered in chronological time. The note(s) can indicate medical observations related to the patient, e.g., times at which particular events occur during the medical procedure. The notes for each patient can be in the form of a note file that includes the medical staff member's notes that were recorded for the patient during the medical procedure. Similarly, the annotations for each patient can be in the form of an annotation file that includes each annotation made by an expert neurophysiologist.

The model training system pre-processes the obtained data (304). The model training system can pre-process the data prior to generating the feature values. This pre-processing can include removing any patient identifying information such that the data stored at the model training system cannot be correlated with any actual patient. The pre-processing can also include converting the EEG signal file to a standard machine processable format, e.g., into a data file that includes values for the EEG signals and, for each signal, a time at which the signal was measured and data describing the signal (e.g., the frequency band and channels for the signal).

The pre-processing can also include extracting, for each patient, information about specified events from the note file. For example, the model training system can parse the note file to detect each specified event and extract information about the event from the note file, e.g., extract a time at which each specified event occurred. In some implementations, the model training system can use pattern matching techniques to detect particular events. For example, the model training system can include, for each relevant event, a set of patterns determined to be ways in which medical staff members indicate the events in the note files. As described above, one example event is a time at which a carotid artery is clamped during a medical procedure.

The pre-processing can also include obtaining, from the annotation file for each patient, the time(s) when a reduced blood flow condition, e.g., ischemia or stroke, was detected. The time can indicate the time at which the condition was detected and/or the time period between the beginning and end of the condition.

The model training system generates feature values for a set of features related to each patient (306). The feature values can represent the EEG signals, times at which specified events occurred, and/or times at which reduced blood flow conditions were detected if any were detected.

To generate the feature values, the model training system can perform the following operations for the data for each patient. The model training system can identify and extract EEG signals for a first time period that represents normal EEG signals for the patient. For example, the first time period can be a specified time period before a clamp is placed on a carotid artery. In a particular example the first time period can be a three-minute, four-minute, or five-minute time period before the clamp is applied. Other appropriate time periods can also be used. The model training system can correlate the time at which the clamp was applied with timestamps or other time indicators for the EEG signals to identify the appropriate bounds of the EEG signals for the first time period.

The model training system also identifies and extracts, for each patient, EEG signals for a second time period starting from when the clamp is applied. The second time period can be the 10-minute period after the clamp was applied, or another appropriate time period. This 10-minute time period can be partitioned into 20 second sub-time periods, or other appropriate sub-time periods, to generate partitioned EEG signals. These sub-time periods can be overlapping. For example, the model training system can generate a 20-second partition of EEG signals for each second during the relevant time period for training the machine learning model. In this example, there can be a first partition for time 0:00 (zero minutes and zero seconds after the clamp is applied to time 0:20 (zero minutes and 20 seconds after the clamp is applied). There can be a second partition for time 0:01 to 0:21, a third partition for 0:02 to 0:22, and so on. Other time increments and time periods for each partition can also be used.

The model training system can convert the EEG signals for the first time period and for each sub-time period from the frequency domain to the power domain using Fast Fourier Transform (FFT). The model training system can also normalize the power for each sub-time period using the power calculated for the first time period. The model training system can normalize the power for each sub-time period using the first 20 seconds of the EEG (e.g., before the clamp is applied) to calculate power and the power of subsequent sub-time periods are normalized in comparison to the first 20 seconds.

The model training system generates feature values for the EEG converted and normalized EEG signals. In some implementations, the model training system generates feature values corresponding to 122 different features for each sub-time period from the partitioned EEG signal for the sub-time period. In this example, the model training system can generate 11 feature values corresponding to features for each of a set of frequency bands, e.g., for a delta frequency band, a theta frequency band, an alpha frequency band, a beta frequency band, and a gamma frequency band. Table 1 below shows the 11 features for the delta frequency range. The model training system can generate the same or similar features for each other frequency band. Further, the model training system can generate the same or similar features across all the five frequency bands combined.

TABLE 1

| Feature | Description |
|---|---|
| Delta_pwr_F3-T3 | Power of delta frequency band from channel F3-T3 |
| Delta_pwr_F3-P3 | Power of delta frequency band from channel F3-P3 |
| Delta_pwr_P3-O1 | Power of delta frequency band from channel P3-O1 |
| Delta_pwr_T3-O1 | Power of delta frequency band from channel T3-O1 |
| Delta_pwr_1 | Averaged power of left hemisphere channels |
| | (F3-T3, F3-P3, P3-O1, and T3-O1) |
| Delta_pwr_F4-T4 | Power of delta frequency band from channel F4-T4 |
| Delta_pwr_F4-P4 | Power of delta frequency band from channel F4-P4 |
| Delta_pwr_P4-O2 | Power of delta frequency band from channel P4-O2 |
| Delta_pwr_T4-O2 | Power of delta frequency band from channel T4-O2 |
| Delta_pwr_2 | Averaged power of right hemisphere channels |
| | (F4-T4, F4-P4, P4-O2, and T4-O2) |
| Delta_pwr_0 | Averaged power of all channels |

The model training system can also generate feature values corresponding to features related to ratios between the alpha frequency band and the delta frequency band for each sub-time period. These alpha to delta ratio (ADR) features can include a ratio between each power value from the 11 features of the alpha frequency band and the corresponding power value from the 11 features of the delta frequency band. Table 2 below shows the ADR features.

TABLE 2

| Feature | Description |
|---|---|
| ADR_F3-T3 | ADR from channel F3-T3 |
| ADR_F3-P3 | ADR from channel F3-P3 |
| ADR_P3-O1 | ADR from channel P3-O1 |
| ADR_T3-O1 | ADR from channel T3-O1 |
| ADR_1 | Averaged ADR from left hemisphere channels |
| | (F3-T3, F3-P3, P3-O1, and T3-O1) |
| ADR_F4-T4 | ADR from channel F4-T4 |
| ADR_F4-P4 | ADR from channel F4-P4 |
| ADR_P4-O2 | ADR from channel P4-O2 |
| ADR_T4-O2 | ADR from channel T4-O2 |
| ADR_2 | Averaged ADR from right hemisphere channels |
| | (F4-T4, F4-P4, P4-O2, and T4-O2) |
| ADR_0 | Averaged ADR from all channels |

Similarly, the model training system can generate, as feature values, beta to delta ratio (BDR) signals which represent the power of the beta frequency band divided by the power of the delta frequency band for each sub-time period. These BDR features can similarly include a ratio between each power value from the 11 features of the beta frequency band and the corresponding power value from the 11 features of the delta frequency band, resulting in 11 additional features for each sub-time period. That is, the BDR features can be the same as the ADR features, but replacing the alpha frequency band values with the beta frequency band values in the calculations.

The model training system can also generate features related to the ratios between the alpha frequency band plus the beta frequency band to the delta frequency band plus the theta frequency band for each sub time-period. These alpha plus beta to delta plus theta ratios (ABDTRs) can be calculated as the sum of the 11 power values of the alpha and beta frequency bands divided by the sums of the corresponding 11 power values of the delta and theta frequency bands, resulting in 11 additional features for each sub-time period.

The model training system can also generate features related to amplitude-integrated EEG (aEEG). The aEEG feature value is calculated as the difference between the highest and lowest voltage among all of the frequency bands. Similar to the ADR features, 11 aEEG features are created for each sub-time period. That is, an aEEG feature is created for each of the 11 power features shown in Table 1 by calculating the difference between the highest voltage for that feature (e.g., row 1 of Table 1) among the five frequency bands and the lowest voltage for that feature (e.g., row 1 of Table 1) among the five frequency bands.

The model training system can also generate features related to the Spectral Edge Frequency 90% (SEF90). The SEF90 feature value can be calculated as the frequency at which 90% of the EEG power for the sub-time period is at or lower than this frequency. Similar to the ADR features, 11 SEF90 features are created for each sub-time period. That is, an SEF90 feature value is calculated for each of the 11 features of Table 1, but using the power values across all frequency bands.

The model training system can also generate features related to pairwise derived Brain Symmetry Index (pdBSI). The pdBSI feature values can be calculated between a pair of corresponding channels on the left and right hemispheres as the difference between the powers divided by the sum of the powers. Five example pdBSI features are shown in Table 3 below.

TABLE 3

| Feature | Description |
|---|---|
| pdBSI_F3-F4 | pdBSI from electrodes F3 and F4 |
| pdBSI_P3-P4 | pdBSI from electrodes P3 and P4 |
| pdBSI_T3-T4 | pdBSI from electrodes T3 and T4 |
| pdBSI_O1-O2 | pdBSI from electrodes O1 and O2 |

Overall, in this example, the model training system can calculate 122 feature values for each sub-time period for each patient. The model training system can also create a target for each sub-time period. This target has a positive value if the reduced blood flow condition occurred during the sub-time period and a negative value if the reduced blood flow condition did not occur during the sub-time period, as obtained from the annotation file for the patient.

The model training system splits the feature values into a training set and a testing set (308). The model training system can split the feature values into training data and testing data. For example, the model training system can split the feature values for a first subset of the patients into training data and the feature values for a second subset of the patients into testing data. As described above, the subsets can be split in various ways, such as 50% of the patients in each subset, 75% in the first subset and 25% in the second subset, or another appropriate way.

The model training system trains one or more machine learning models (310). The model training system can train the machine learning model(s) using the feature values and the target for each patient in the training data. In some implementations, the model training system trains multiple different types of machine learning models using the training data. For example, the model training system can train artificial neural networks, logistic regression models, naïve Bayes classifiers, support vector classifiers, and/or random forest classifiers. These models can be trained using supervised learning algorithms, as described above. After training the models, the model training system can fine tune the models. For example, two models with different levels of performance, such as a highly sensitive model and a highly specific model, can be used in tandem. As another example, the probabilistic output of a model can be translated into risk levels, such as low, moderate, and high risk of reduced blood flow state.

The model training system evaluates the trained machine learning models (312). For example, as described above, the model training system can apply each model on the testing data and can determine the area under the ROC curve for each machine learning model. The evaluation can include determining which trained machine learning model has the best combination of performance based on the area under the ROC curve, sensitivity, and specificity.

The model training system can store and/or provide one or more of the trained machine learning models (314). For example, the model training system can select the trained machine learning model that has, according to specified optimization criteria, an optimized or best combination of performance, sensitivity, and specificity and store the selected model for use in blood condition monitoring devices. In some implementations, the model training system can deploy the model to the devices and/or update the models deployed on the devices using a newly trained machine learning model. For example, the model training system can receive updated patient data from time to time, e.g., periodically, and retrain or update the machine learning models using the updated patient data.

In some implementations, the model training system evaluates deployed machine learning models, e.g., periodically. For example, the model training system can provide input data to the machine learning model and compare the output to labels for the input data. If the performance of the model is low (e.g., lower than a threshold) or trending downwards, the model training system can retrain one or more machine learning models and select a best performing model to deploy to the blood flow condition monitoring device.

FIG. 4 is a flow diagram of an example process 400 for detecting reduced blood flow conditions in patients using a trained machine learning model. In some implementations, the example process 400 can be performed by a blood flow condition monitoring device, such as the blood flow condition monitoring device 150 of FIG. 1. Operations of the process 400 can also be implemented as instructions stored on non-transitory computer readable media, and execution of the instructions by one or more data processing apparatus can cause the one or more data processing apparatus to perform the operations of the process 400. For brevity, the process will be described in terms of a monitoring device.

The monitoring device receives patient data (402). The monitoring device can receive the patient data in real-time, e.g., while the patient is undergoing a medical procedure. This enables the monitoring device to detect a reduced blood flow condition, e.g., ischemia or stroke, in real-time so that medical staff can intervene and prevent and/or reduce the impact of the condition. As described above, the patient data can include EEG signals received from an EEG device. In some cases, the patient data can include medical staff notes that indicate patient observations.

The monitoring device generates feature values of features based on the patient data (404). This can include pre-processing operations that are similar to the pre-processing operations performed on patient data prior to training the machine learning models. For example, the monitoring device can remove patient identifying information, and convert EEG files to a standard machine processable format. In some cases, the monitoring device can extract information about specified events from note files. As described above, a data formatting engine can use memory management techniques to process the data rapidly to ensure quick detection of reduced blood flow conditions.

The monitoring device can generate the same (or similar) features as those used to train the machine learning model. For example, the monitoring device can generate the 122 EEG feature values described above for training the machine learning model. However, the monitoring device can use the same or a different time period for the feature values. For example, rather than calculating the feature values for 20 second sub-time periods, the monitoring device can use a shorter time period, e.g., calculating the feature values for every 500 milliseconds, every second, every two seconds, every five seconds, etc.

In some implementations, the monitoring device generates feature values for each 20 second (or other) time period each second (or based on a different interval). In this example, each second the monitoring device would generate feature values based on the EEG signals for the 20 second period leading up to the current time. In this way, each 20 second time period is evaluated in real-time to detect a reduced blood flow condition.

The monitoring device provides the feature values for the EEG signals as input to a trained machine learning model (406). In some cases, the monitoring device can provide feature values for the specified event extracted from the notes as input to the trained machine learning model. The monitoring device applies the trained machine learning model to the feature values. For example, the monitoring device can apply the feature values to the machine learning model each time new feature values are generated. The trained machine learning model is trained to output an indication of whether the patient has (e.g., is experiencing) the reduced blood flow condition in one of the patient's organs based on changes to the EEG signals over time during the medical procedure.

The monitoring device receives a machine learning output (408). The machine learning output indicates a likelihood that, or whether, the patient has (e.g., is experiencing) the reduced blood flow condition in one or more of the patient's organs. For example, the machine learning output can be a score that indicates the likelihood that the patient has the reduced blood flow condition in one or more of the patient's organs.

The monitoring device provides an output that indicates whether the patient has (e.g., is experiencing) the reduced blood flow condition in one of the patient's organs (410). For example, the monitoring device can compare the score to one or more thresholds. If the score satisfies (e.g., meets or exceeds) a threshold indicative of a reduced blood flow condition, the monitoring device can output that the patient is experiencing a reduced blood flow condition. If the score is less than a threshold indicative of a normal or non-reduced blood flow condition, the monitoring device can output that the patient is not experiencing a reduced blood flow condition.

In some implementations, the output (e.g., the score or condition determined based on the score) is continuously displayed, e.g., by a monitor connected to the monitoring device, to medical staff in an operating room in which the medical procedure is being performed. In some implementations, if the reduced blood flow condition is detected, the monitoring device sends a notification, e.g., alert, to medical staff notifying them that the condition is occurring. For example, the monitoring device can generate an alert in the operating room and/or send an alert to devices of a response team in response to detecting the condition.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, general or special purpose microprocessors or both, or any other kind of central processing unit. Generally, a central processing unit will receive instructions and data from a read-only memory or a random-access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

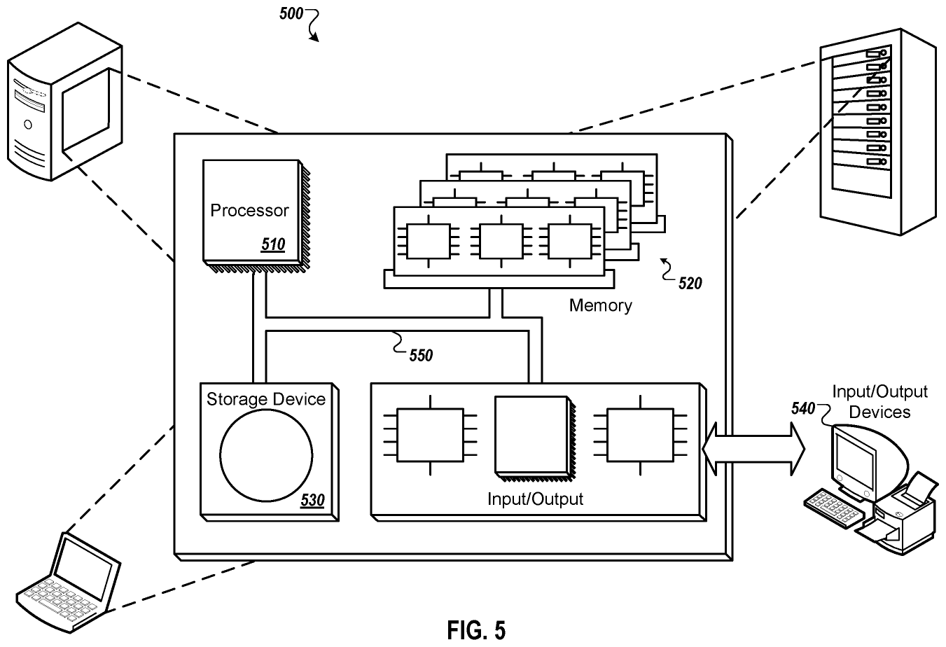

An example of one such type of computer is shown in FIG. 5, which shows a schematic diagram of a generic computer system 500. The system 500 can be used for the operations described in association with any of the computer-implemented methods described previously, according to one implementation. The system 500 includes a processor 510, a memory 520, a storage device 530, and an input/output device 540. Each of the components 510, 520, 530, and 540 are interconnected using a system bus 550. The processor 510 is capable of processing instructions for execution within the system 500. In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the system 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the system 500. In one implementation, the storage device 530 is a computer-readable medium. In various different implementations, the storage device 530 may be a floppy disk device, a hard disk device, an optical disk device, or a tape device.

The input/output device 540 provides input/output operations for the system 400. In one implementation, the input/output device 540 includes a keyboard and/or pointing device. In another implementation, the input/output device 540 includes a display unit for displaying graphical user interfaces.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more data processing apparatus, the method comprising:
receiving patient data comprising a set of electroencephalography (EEG) signals generated by an EEG device measuring brain function of the patient;
generating, using the EEG signals, a set of feature values for features of the patient;
providing the feature values as input to a trained machine learning model that has been trained to detect reduced blood flow conditions of patients;
receiving, as a machine learning output of the trained machine learning model, an indication of whether the patient has the reduced blood flow condition; and
providing the indication of whether the patient has the reduced blood flow condition.

2. The method of claim 1, wherein the reduced blood flow condition comprises one of ischemia or a stroke.

3. The method of claim 1, comprising determining that the patient has the reduced blood flow condition based on the machine learning output, wherein providing the indication of whether the patient has the reduced blood flow condition comprises sending an alert to one or more medical professionals.

4. The method of claim 1, wherein the trained machine learning model is trained based on historical patient data for a plurality of patients, the historical data for each individual patient in the plurality of patients comprising sequences of EEG signals for the individual patient that were monitored during a medical procedure being performed on the individual patient, one or more medical professional notes generated by a medical professional during the medical procedure, and annotations indicating when, relative to the sequences of EEG signals, a reduced blood flow condition was detected for the individual patient during the medical procedure.

5. The method of claim 1, wherein the feature values comprise a set of ratios between power values of a first frequency band of the EEG signals and corresponding power values of a second frequency band of the EEG signals.

6. The method of claim 1, wherein the feature values comprise a ratio between (i) a first sum of a first power value of a first frequency band of the EEG signals and a second power value of a second frequency band of the EEG signals and (ii) a second sum of a third power value of a third frequency band of the EEG signals and a fourth power value of a fourth frequency band of the EEG signals.

7. The method of claim 1, wherein the feature values comprise a difference between a highest voltage among the EEG signals and a lowest voltage among the EEG signals.

8. The method of claim 1, wherein the feature values comprise a particular frequency at which a specified percentage of power values of the EEG signals is at or lower than the particular frequency.

9. The method of claim 1, wherein generating the feature values comprises generating a set of feature values in real-time for each second of EEG signals received from an EEG device connected to the patient.

10. The method of claim 9, wherein each set of feature values comprises feature values for a time period beginning a specified amount of time before the second for which the set of feature values is generated.

11. The method of claim 10, wherein the specified amount of time is 20 seconds.

12. The method of claim 1, wherein the patient data comprises a set of data files that each include different formats of data including multiple data files comprising data for the set of EEG signals.

13. The method of claim 12, comprising preprocessing the data of each data file to convert the data of each data file to a same standard format.

14. The method of claim 13, comprising:
maintaining each data file in an open state throughout a time period in which the patient is being monitored; and
for each data file, continuously or periodically scanning memory locations at which data for each data file is stored to acquire any new data written to the memory locations.

15. The method of claim 14, wherein continuously or periodically scanning memory locations at which data for each data file is stored to acquire any new data written to the memory locations comprises monitoring a flag that indicates an end of file location in memory for the data file.

16. A computer-implemented system, comprising:
one or more computers; and
one or more computer memory devices interoperably coupled with the one or more computers and having tangible, non-transitory, machine-readable media storing one or more instructions that, when executed by the one or more computers, perform operations comprising:
receiving patient data comprising a set of electroencephalography (EEG) signals generated by an EEG device measuring brain function of the patient;
generating, using the EEG signals, a set of feature values for features of the patient;
providing the feature values as input to a trained machine learning model that has been trained to detect reduced blood flow conditions of patients;
receiving, as a machine learning output of the trained machine learning model, an indication of whether the patient has the reduced blood flow condition; and
providing the indication of whether the patient has the reduced blood flow condition.

17. The computer-implemented system of claim 16, wherein the reduced blood flow condition comprises one of ischemia or a stroke.

18. The computer-implemented system of claim 16, wherein the operations comprise determining that the patient has the reduced blood flow condition based on the machine learning output, wherein providing the indication of whether the patient has the reduced blood flow condition comprises sending an alert to one or more medical professionals.

19. The computer-implemented system of claim 16, wherein the trained machine learning model is trained based on historical patient data for a plurality of patients, the historical data for each individual patient in the plurality of patients comprising sequences of EEG signals for the individual patient that were monitored during a medical procedure being performed on the individual patient, one or more medical professional notes generated by a medical professional during the medical procedure, and annotations indicating when, relative to the sequences of EEG signals, a reduced blood flow condition was detected for the individual patient during the medical procedure.

20. A non-transitory, computer-readable medium storing one or more instructions executable by a computer system to perform operations comprising:
receiving patient data comprising a set of electroencephalography (EEG) signals generated by an EEG device measuring brain function of the patient;
generating, using the EEG signals, a set of feature values for features of the patient;
providing the feature values as input to a trained machine learning model that has been trained to detect reduced blood flow conditions of patients;
receiving, as a machine learning output of the trained machine learning model, an indication of whether the patient has the reduced blood flow condition; and
providing the indication of whether the patient has the reduced blood flow condition.
